(19) United States
(12) Patent Application Publication
(10) Pub. No.: US 2021/0133429 A1
(43) Pub. Date: May 6, 2021

(54) AUGMENTED REALITY SYSTEM FOR MEASUREMENT AND THERAPEUTIC INFLUENCE OF MENTAL PROCESSES

(71) Applicant: GENERAL ELECTRIC COMPANY, Schenectady, NY (US)

(72) Inventor: Peter W. Lorraine, Niskayuna, NY (US)

(21) Appl. No.: 16/671,814

(22) Filed: Nov. 1, 2019

(51) Int. Cl. G06K 9/00; A61B 5/16; G06T 17/20

(52) U.S. Cl. CPC G06K 9/00261; A61B 5/165; G06K 9/00375; G06K 9/00308; G06T 17/20; G06K 9/00281

(57) ABSTRACT

According to some embodiments, a system, method and non-transitory computer-readable medium are provided to measure mental processes of a subject including receiving a first image representative of a face of a subject from an image capture device; determining one or more first facial geometry features from the first image; comparing the one or more first facial geometry features to one or more corresponding target facial geometry features; modulating the one or more first facial geometry features based upon the comparing of the one or more first facial geometry features to the one or more corresponding target facial geometry features to generate a first modulated image; displaying the first modulated image in a display device; receiving a second image of the face of the subject, the second image representative of a response of the subject to viewing the first modulated image; and analyzing the second image to determine a mental process associated with the subject.

### BACKGROUND

Mental processes and states are notoriously difficult to measure due to their subjective nature, yet are linked to important health states of a person. Existing approaches to measuring mental processes have involved administering questionnaires to subjects. However, questionnaires require time to administer and may lead the subject to provide answers matched to a perceived goal. Another existing approach involves using functional magnetic resonance imaging (MRI) to identify activity maps within a subject's brain that may be associated with depression, anxiety, or other mental states of interest. However, MRIs are expensive to administer. It may also be desirable to provide a therapeutic influence upon a subjects mental processes to positively influence the subject's mental state.

Existing approaches to measure and provide a therapeutic influence upon mental processes may not adequately address these problems.

It would be desirable to provide systems and methods to improve measurement and therapeutic influences of mental processes of a subject to provide more accurate and efficient results.

### SUMMARY

According to some embodiments, a computer-implemented method includes receiving a first image representative of a face of a subject from an image capture device, determining one or more first facial geometry features from the first image, and comparing the one or more first facial geometry features to one or more corresponding target facial geometry features. The method further includes modulating the one or more first facial geometry features based upon the comparing of the one or more first facial geometry features to the one or more corresponding target facial geometry features to generate a first modulated image, and displaying the first modulated image in a display device. The method further includes receiving a second image of the face of the subject, the second image representative of a response of the subject to viewing the first modulated image, and analyzing the second image to determine a mental process associated with the subject.

According to some embodiments, a system includes an image capture device, a display device, and a processing device. The processing device is configured to receive a first image representative of a face of a subject from the image capture device, determine one or more first facial geometry features from the first image, and compare the one or more first facial geometry features to one or more corresponding target facial geometry features. The processing device is further configured to modulate the one or more first facial geometry features based upon the comparing of the one or more first facial geometry features to the one or more corresponding target facial geometry features to generate a first modulated image, and display the first modulated image in the display device. The processing device is further configured to receive a second image of the face of the subject, the second image representative of a response of the subject to viewing the first modulated image, and analyze the second image to determine a mental process associated with the subject.

According to some embodiments, a non-transitory computer-readable medium storing instructions that, when executed by a computer processor, cause the computer processor to perform a method including receiving a first image representative of a face of a subject from an image capture device, determining one or more first facial geometry features from the first image, and comparing the one or more first facial geometry features to one or more corresponding target facial geometry features. The method further includes modulating the one or more first facial geometry features based upon the comparing of the one or more first facial geometry features to the one or more corresponding target facial geometry features to generate a first modulated image, and displaying the first modulated image in a display device. The method further includes receiving a second image of the face of the subject, the second image representative of a response of the subject to viewing the first modulated image, and analyzing the second image to determine a mental process associated with the subject.

According to some embodiments, a computer-implemented method includes receiving a first image representative of a body portion of a subject from an image capture device, determining one or more first geometry features from the first image, and comparing the one or more first geometry features to one or more corresponding target geometry features. The method further includes modulating the one or more first geometry features based upon the comparing of the one or more first geometry features to the one or more corresponding target geometry features to generate a first modulated image, and displaying the first modulated image in a display device. The method further includes receiving a second image of the body portion of the subject wherein the second image is representative of a response of the subject to viewing the first modulated image. The method further includes analyzing the second image to determine a mental process associated with the subject.

Some technical effects of some embodiments disclosed herein are improved systems and methods to determine mental processes of a subject in a new and quantitative manner. Drug discovery, for example for antidepressant medications, may be enabled by more accurate or rapid determinations of symptoms without requiring the use of questionnaires. In some embodiments, the systems and method described herein may be used as a diagnostic tool, therapeutic tool and/or research tool to determine and beneficially influence mental processes of subjects. Some embodiments may be implemented using low-cost hardware and/or software platforms that are portable to allow the ability to create quantifiable data than can be uploaded to healthcare professionals to enable use in either a home or medical-care provider settings.

### BRIEF DESCRIPTION OF THE DRAWINGS

FIG. 1 is a high-level block diagram of a system that may be provided in accordance with some embodiments.

FIG. 2 represents processes that may be performed by some or all of the elements of the system described with respect to FIG. 1 in accordance with some embodiments.

FIG. 3 represents operations that may be performed by some or all of the elements of the system described with respect to FIG. 1 for modulation of a captured image of a subject in accordance with some embodiments.

FIG. 4 represents processes of a measurement/interaction cycle that may be performed by some or all of the elements of the system described with respect to FIG. 1 in accordance with some embodiments.

FIG. 5 illustrates a process 500 that might be performed by some of all of the elements of the system 100 described with respect to FIG. 1 in accordance with some embodiments.

FIG. 6 is a block diagram of an augmented reality processing system for measurement and therapeutic influences of mental processes of a subject according to some embodiments of the present invention.

### DETAILED DESCRIPTION

In the following detailed description, numerous specific details are set forth in order to provide a thorough understanding of embodiments. However, it will be understood by those of ordinary skill in the art that the embodiments may be practiced without these specific details. In other instances, well-known methods, procedures, components and circuits have not been described in detail so as not to obscure the embodiments.

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One or more embodiments provide for a device that functions as a “mirror” to capture an image of a subject with a camera and produce a subtly altered reflection image of a subject in a display to induce a change in self-image. The device measures the resulting change in detailed body and face geometry of the subject in response to the altered reflection image, and characterizes the resulting response for various stimulus alterations. The device uses the measurements and characterizations to determine underlying mental process dynamics of the subject. The device may use these measurements to characterize underlying conditions of the subject and/or serve as a diagnostic tool for multiple conditions. In one or more embodiments, the response to stimulus can also be used as a therapeutic tool as part of a treatment or wellness application.

Although various embodiments described herein are directed to facial geometry of a subject, it should be understood that other embodiments may be directed to one or more other portions of a body of subject that may relate to a “self-image” such as a changed position of a hand that may elicit a response from the subject. In one or more embodiments, a first image representative of a body portion of a subject is received from an image capture device, and one or more first geometry features are determined from the first image. In particular embodiments, the body portion may include a face or an arm position of the subject. The one or more first geometry features are compared to one or more corresponding target geometry features, and the one or more first geometry features are modulated based upon the comparing of the one or more first geometry features to the one or more corresponding target geometry features to generate a first modulated image. The first modulated image is displayed in a display device. A second image of the body portion of the subject is received in which the second image is representative of a response of the subject to viewing the first modulated image. The second image is analyzed to determine a mental process associated with the subject.

Another embodiment may further include determining one or more second geometry features from the second image, comparing the one or more second geometry features to the one or more corresponding target geometry features, and determining the mental process of the subject based upon the comparing of the one or more second geometry features to the one or more corresponding target geometry features. In a particular embodiment, modulating the one or more first geometry features includes morphing the first geometry features towards the one or more target geometry features by a predetermined amount.

A key unique aspect of one or more embodiments is to use a trusted source of information such as a “mirror” image to influence self-perception in a novel and more effective way. The change in the “mirror” image is accomplished by careful measurement of the human source subject and introducing subtle modulations, such as an increased smile, to the image. In one or more embodiments, the amplitude and dynamics of the subject's response to the modulated image is characterized. The resulting response from the human subject is measured to determine mental processes of the subject and/or cause a therapeutic influence upon such mental processes. Such measurements are quantitative and reproducible, and do not require spoken or questionnaire responses from the subject. In a particular application, a system measures changes in the subject's response that are associated with particular mental conditions, such as depression or post-traumatic stress disorder (PTSD). Positive responses, if observed, may be used as part of therapeutic influences on these conditions as well as used as a tool to research the evolution of mental conditions, decision making, and therapy.

In one or more embodiments, a subtly modified image of a subject that appears as an accurate representation of the subject and is trusted at a fundamental level. The modification to the image is perceived by the subject indirectly and the subject “accommodates” the perceived discrepancy by altering the appearance of the subject in a small way. As an example, an image that appears to smile slightly more than the subject might influence the subject to increase the amount that the subject is smiling in a manner analogous to yawn-contagion. In one or more embodiments, a system measures the amount of the response to the modification and may provide insight to underlying mental process dynamics of the subject. In an example use case, a subject with low emotional affect due to depression might respond in a different manner than a more typical subject.

One or more embodiments may facilitate easy and more continuous monitoring of the coupling between stimulus and response by observing the modulated “mirror” image and the subject's response. A more accurate gauge of mental processes may aid in evaluating treatment progress or drug efficacy in a study. In addition, if a subject responds to a stimulus, the evoked response may be incorporated into therapy or general wellness treatments. An ability to reduce undesired emotional states, for example, anger or fear in an ill patient, may support alternatives to medication as part of a treatment protocol.

Although some examples are described with respect to emotional responses, the principles described herein may also be applicable to any problem in which a self-image modification may influence a subject's physical performance such as involuntary motion, tremor modification, balance, or other issues.

In some embodiments, the device and its response can be optimized for an amount and temporal characteristic of any modulation of the subject image with feedback provided either directly or by measurement of the subject geometry and associated image, as well as including other feedback mechanisms such as functional magnetic resonance imaging (fMRI), electroencephalogram (EEG), or other observational tools.

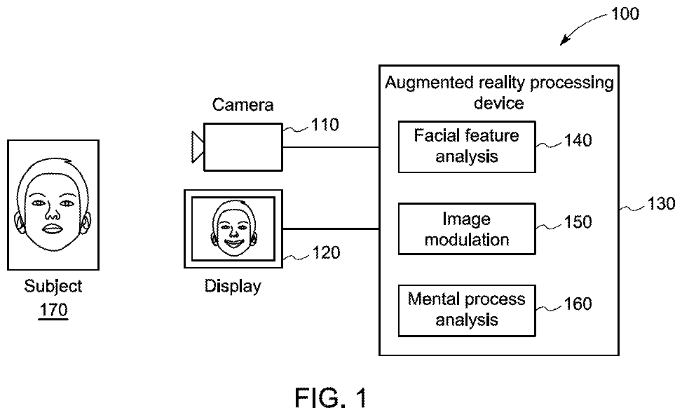

FIG. 1 is a high-level architecture of a system 100 in accordance with some embodiments. The system 100 includes a camera device 110 and display device 120 in communication with an augmented reality processing device 130. Augmented reality processing device 130 includes a facial feature analysis component 140, an image modulation component 150, and a mental process analysis component 160. In some embodiments, the camera device 110, display device 120, and augmented reality processing device 130 are integrated into a single component such as a smartphone or augmented reality googles. In still other embodiments, one or more of camera device 110, display device 120, and augmented reality processing device 130 may be separate components.

Camera device 110 is configured to capture an image of a subject 170 and provide the captured image to augmented reality processing device 130. In one or more embodiments, subject 170 is a human subject and the captured image includes at least a portion of the face of the human subject. The facial feature analysis component 140 is configured to analyze the captured image to extract one or more observed facial features of the captured image. In particular embodiments, facial features may include, but are not limited to, one or more of mouth geometry, eye geometry, facial muscle geometry, eyebrow geometry, or other facial geometry of the subject 170 that may be indicative of a mental process associated with the subject 170. For example, mouth geometry associated with a degree of smiling may be indicative of an underlying emotional state or other mental process of the subject 170.

Facial features analysis component 140 is further configured to compare the observed facial features of the captured image to one or more facial features associated with an expected and/or desired mental process to determine differences (e.g., differences in amplitude and/or dynamics) between the observed facial features and the expected facial features.

Image modulation component 150 is further configured to determine one or more facial feature modulations to the captured image to alter the appearance of the facial features of the subject 170 in the captured image, and apply the one or more facial feature modulations to the captured image to generate a modulated image including a modified appearance of the subject 170. In an example, the modulated image may include a representation of the subject 170 appearing to exhibit a greater degree of smiling than the captured image of the subject 170. In one or more embodiments, the image modulation component 150 uses a forcing function to determine the altered appearance that is based upon a difference between the observed state of the facial features of the subject 170 and an expected state of the facial features of the subject 170.

The image response can be described numerically and derived from geometric parameters that can be measured reliably. For example, a smile angle can be parametrized to be in the range of 0-1 as can other facial geometry features such as eye squint, mouth angle, right eye open and others. In the same way, in certain embodiments the asserted modulation can also be described numerically with the same parameter set. If a is defined as the change in the response geometry to a modulation εⱼ, the response to the modulation can be related by the simplified equation:

σᵢ=Cᵢⱼε_{I}

where Cᵢⱼ is a tensor relating the two quantities. In certain embodiments, more sophisticated models including time dependency may be used, but the key observation is that all of the values can be determined numerically from visual observation. The same model can be extended to more general body geometry. The specific changes in Cᵢⱼ over time in the same patient, or across patients may capture differences in internal mental states.

Augmented reality processing device 130 is further configured to send the modulated image to display device 120. Display device 130 is configured to display the modulated image to the subject 170. Responsive to viewing the modulated image, the subject 170 may be induced to change or modify a mental process such as experience an increase in happiness. The change or medication of the mental process may further cause the subject to change one or more of the facial features associated with the mental process such as increasing a degree of smiling by the subject 170.

Camera device 110 is further configured to capture a second image of the subject 170 representative of the facial response of the subject 170 to viewing the modulated image and provide the second captured image to facial feature analysis component 140. Facial feature analysis component 140 is further configured to extract a new set of facial features from the second captured image. Facial feature analysis component 140 is further configured to determine a change in the facial features of the subject 170 between the initial captured image of the subject 170 and the second captured image of the subject 170. In a particular embodiment, facial feature analysis component 140 determines the change in the facial features between the initial captured image by measuring an amplitude and dynamics of the change of facial features between the initial captured image and the second captured image.

Mental process analysis component 160 is configured to measure one or more internal mental processes of the subject 170 based upon the response of the subject 170 to the modulated image. In one or more embodiments, the response of the subject 170 is correlated with a mental condition of the subject 170 such as readiness, resilience, trainability, depression, or anxiety. In particular embodiments, a degree of response of the subject 170 to the modulated image may be indicative of particular mental processes.

In an example operation, the facial features of an initial captured image of the subject 170 may exhibit a “neutral” characteristic in which the subject 170 is not smiling or frowning. Augmented reality processing device 130 may generate a modulated image of the subject 170 in which the subject 170 is smiling, and display the modulated image to the subject 170 within the display device 120. Augmented reality processing device 130 may further receive a second captured image of the subject 170 that is indicative of a response to viewing the modulated image by the subject 170. If the facial geometry of the subject 170 exhibits no change or a change below a predetermined threshold value responsive to viewing the modulated image, the mental process analysis component 160 may determine that the subject 170 possesses one or more undesirable internal mental processes or states that are indicative of a reduced response to a stimulus.

If the facial geometry of the subject 170 exhibits a change above the predetermined threshold value responsive to viewing the modulated image, the mental process analysis component 160 may determine that the subject 170 is responsive to the modulated image possesses one or more desirable internal mental processes. For example, if the modulated image depicts the subject 170 as smiling and the subject 170 responds to viewing the modulated image by increasing an amount of smiling, mental process analysis component 160 may determine that subject 170 possesses a positive mental process.

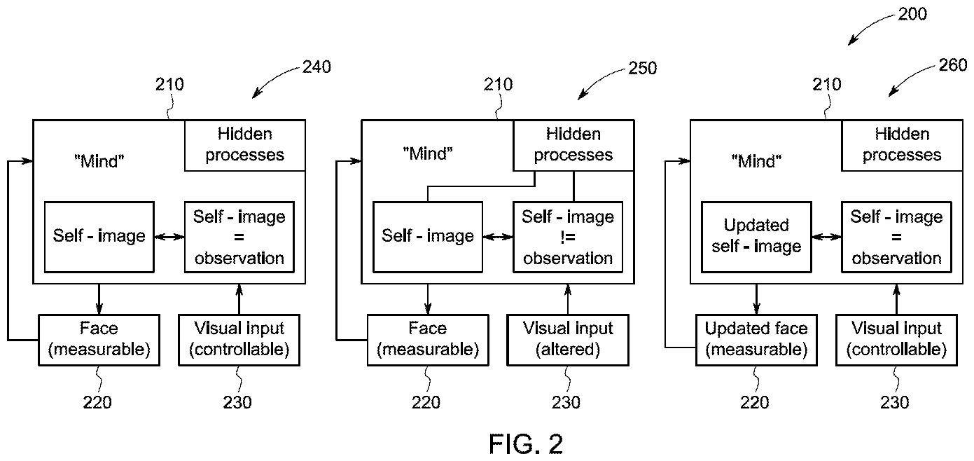

FIG. 2 represents processes 200 that may be performed by some or all of the elements of the system 100 described with respect to FIG. 1 in accordance with some embodiments. FIG. 2 illustrates operations for probing hidden mental processes of a mind 210 of a subject using measurable inputs and outputs. In the illustrated embodiment, an output 220 is represented as a captured image of a face of the subject, and an input 230 is represented as an image of the subject generated by augmented reality processing device 130. Under a normal condition 240, one or more facial features of a captured self-image of the subject determined from the captured image are substantially the same as the corresponding facial features of an observation of the displayed image. Under the normal condition 240, no modulation of the captured image may be performed to generate the displayed image such that the displayed image is substantially unchanged from the captured image.

Under a probe condition 250, one or more of the facial features of the captured self-image of the subject does not match the observed image. As a result, the displayed image is visually modulated and displayed to the subject. Under a perturbed condition 260, the subject views the altered image generated under the probe condition 250, and the subject may change facial geometry in response to viewing the altered image. The augmented reality processing device 130 may measure the change in facial geometry of the subject to disclose hidden mental processes of the subject during a diagnostic process. Under an influence process, the updated face changes may alter the hidden mental processes of the subject.

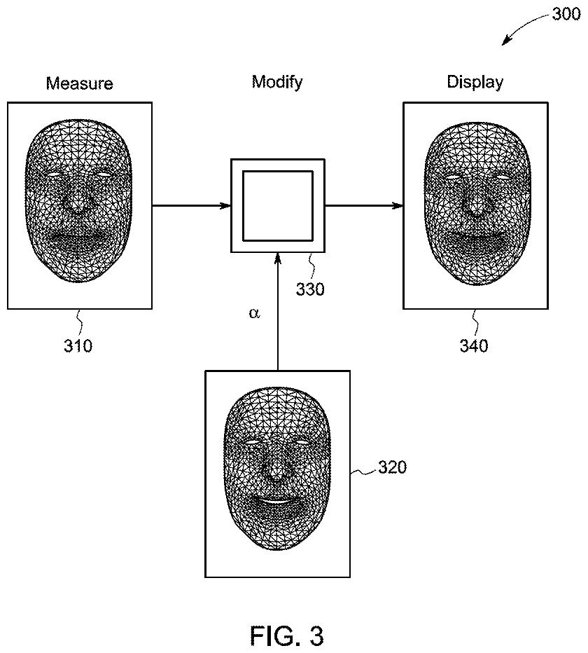

FIG. 3 represents operations 300 that may be performed by some or all of the elements of the system 100 described with respect to FIG. 1 for modulation of a captured image of a subject in accordance with some embodiments. In an operation 310, augmented reality processing device 130 receives an image of the face of a subject from the camera device 110, extracts a shape mesh 310 including a three-dimensional (3D) representation of an initial state of the face of the subject. In particular embodiments, shape mesh 310 may include one or more facial geometry features of the subject such as overall face geometry, nose geometry, mouth geometry, eye geometry, and brow geometry. Based upon the shape mesh 310, the augmented reality processing device 130 determines a target mesh 320 for the subject associated with a target state of the face of the subject.

In the embodiment, the augmented reality processing device 130 modifies the initial state during a modulation operation 330 to morph the initial state of the face of the subject towards the target state by a predetermined perturbation amount a to generate a modulated mesh 340 The augmented reality processing device 130 re-renders the modulated mesh 340 using textures corresponding to textures of the initial image to display a modulated image of the subject. Although FIG. 3 illustrates modulation of a captured image of a subject using 3-D shape meshes, it should be understood that in other embodiments other methods may be used to generate a modulated image from an initial image such as machine learning using a Generative Adversarial Network (“GAN”).

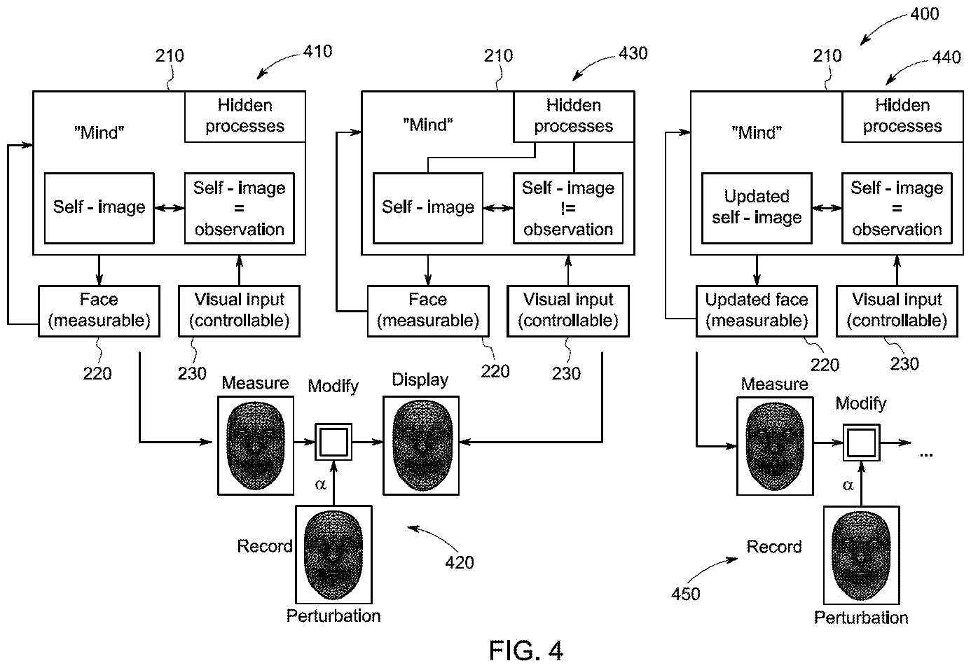

FIG. 4 represents processes of a measurement/interaction cycle 400 that may be performed by some or all of the elements of the system 100 described with respect to FIG. 1 in accordance with some embodiments. FIG. 4 illustrates operations for measuring and interacting with hidden mental processes of a mind 210 of a subject using measurable inputs and outputs. In the illustrated embodiment, an output 220 is represented as a captured image of a face of the subject, and an input 230 is represented as an image of the subject generated by augmented reality processing device 130. In operation 410, one or more facial features of a captured self-image of the subject are measured and modulated using a modulation operation 420 to generate a modulated image.

In operation 430, the modulated image is displayed to the subject. In operation 440, the subject may change facial geometry in response to viewing the altered image. The augmented reality processing device 130 may measure the change in facial geometry of the subject to disclose hidden mental processes of the subject. In operation 450, the captured image of the subject may be further modulated to generate a new modulated image for display to the user. In one or more embodiments, one or more of operations 410-450 may be repeated.

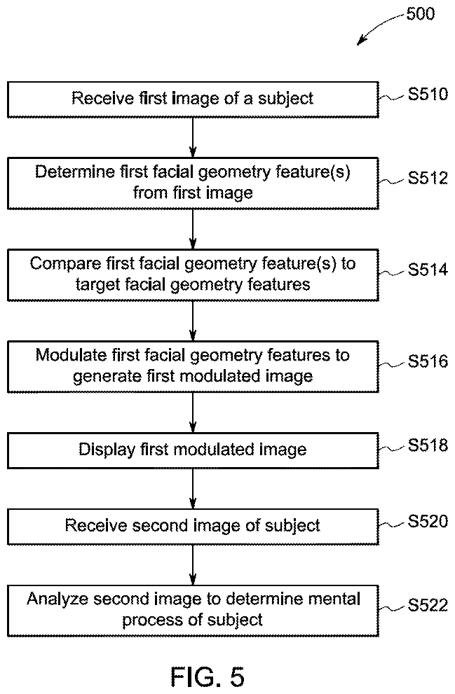

FIG. 5 illustrates a process 500 that might be performed by some of all of the elements of the system 100 described with respect to FIG. 1 in accordance with some embodiments. Process 500, and any other process described herein may be performed using any suitable combination of hardware (e.g., circuit(s)), software or manual means. For example, a computer-readable storage medium may store thereon instructions that when executed by a machine result in performance according to any of the embodiments described herein. In one or more embodiments, the system 100 is conditioned to perform the process 500 such that the system is a special-purpose element configured to perform operations not performable by a general-purpose computer or device. Software embodying these processes may be stored by any non-transitory tangible medium including a fixed disk, a floppy disk, a CD, a DVD, a Flash drive, or a magnetic tape. Examples of these processes will be described below with respect to embodiments of the system, but embodiments are not limited thereto. The flow charts described herein do not imply a fixed order to the steps, and embodiments of the present invention may be practiced in any order that is practicable.

Initially, at S510, a first image representative of a face of a subject is received from an image capture device. At S512, one or more first facial geometry features are determined from the first image. At S514, the one or more first facial geometry features are compared to one or more corresponding target facial geometry features. At S516, the one or more first facial geometry features are modulated based upon the comparing of the one or more first facial geometry features to the one or more corresponding target facial geometry features to generate a first modulated image. At S518, the first modulated image is displayed to the subject in a display device.

At S520, a second image of the face of the subject is received. The second image is representative of a response of the subject to viewing the first modulated image. At S522, the second image is analyzed to determine a mental process associated with the subject.

In one or more embodiments, wherein analyzing the second image includes determining one or more second facial geometry features from the second image, comparing the one or more second facial geometry features to the one or more corresponding target facial geometry features, and determining the mental process of the subject based upon the comparing of the one or more second facial geometry features to the one or more corresponding target facial geometry features.

In one or more embodiments, modulating the one or more first facial geometry features includes morphing the first facial geometry features towards the one or more target facial geometry features by a predetermined amount.

In one or more embodiments, determining the one or more first facial geometry features includes extracting a shape mesh from the first image, and identifying the one or more first facial geometry features from the shape mesh. In an embodiment, the shape mesh includes a three-dimensional representation of an initial state of the subject. In an embodiment, the modulating of the one or more first facial geometry features includes determining a target mesh for the subject associated with a target state of the face of the subject.

In one or more embodiments, the one or more facial geometry features includes at least one of an overall face geometry, a nose geometry, a mouth geometry, an eye geometry, and a brow geometry associated with the subject. In one or more embodiments, the mental process is associated with a mental condition of the subject. In one or more embodiments, the target facial geometry features are associated with a desired mental condition of the subject. In particular embodiments, the subject is a human subject.

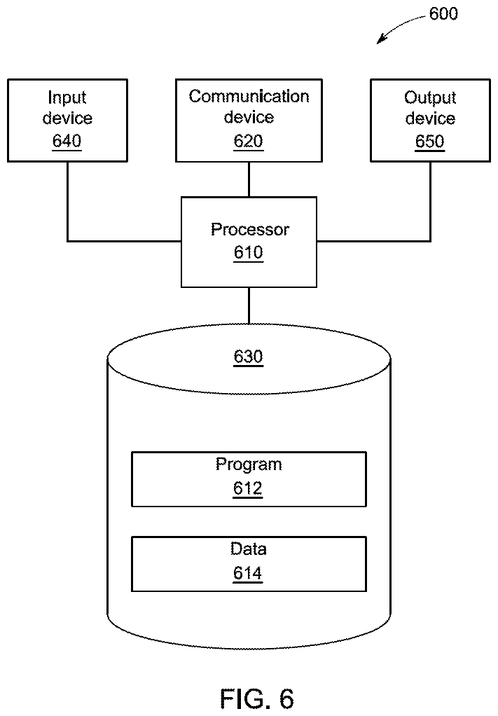

The embodiments described herein may be implemented using any number of different hardware configurations. For example, FIG. 6 is a block diagram of an augmented reality processing system 600 for measurement and therapeutic influences of mental processes of a subject that may be, for example, associated with the system 100 of FIG. 1. The augmented reality processing system 600 comprises a processor 610, such as one or more commercially available Central Processing Units (“CPUs”) in the form of one-chip microprocessors, coupled to a communication device 620 configured to communicate via a communication network (not shown in FIG. 6). The communication device 620 may be used to communicate, for example, with one or more remote data source nodes, user platforms, etc. The augmented reality processing system 600 further includes an input device 640 (e.g., a camera or other image capture device) and/an output device 650 (e.g., a computer monitor to render a display). According to some embodiments, a mobile device, monitoring physical system, and/or PC may be used to exchange information with the augmented reality processing system 600.

The processor 610 also communicates with a storage device 630. The storage device 630 may comprise any appropriate information storage device, including combinations of magnetic storage devices (e.g., a hard disk drive), optical storage devices, mobile telephones, and/or semiconductor memory devices. The storage device 630 stores a program 612 and/or data 614 for controlling the processor 610. The processor 610 performs instructions of the program 612 and thereby operates in accordance with any of the embodiments described herein. For example, the processor 610 may receive captured images associated with a subject. The processor 610 may then perform a process to determine a modulated image, display the modulated image to the subject, and determine a response to the viewing of the modulated image by the subject. The processor may determine and/or influence one or more mental processes of the subject.

The programs 612 may be stored in a compressed, uncompiled and/or encrypted format. The programs 612 may furthermore include other program elements, such as an operating system, clipboard application, a database management system, and/or device drivers used by the processor 610 to interface with peripheral devices.

As used herein, information may be “received” by or “transmitted” to, for example: (i) the augmented reality processing system 600 from another device; or (ii) a software application or module within the augmented reality processing system 600 from another software application, module, or any other source.

The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although specific hardware and data configurations have been described herein, note that any number of other configurations may be provided in accordance with embodiments of the present invention (e.g., some of the information associated with the databases described herein may be combined or stored in external systems). For example, although some embodiments are focused on a power grid, any of the embodiments described herein could be applied to other types of assets, such as damns, wind farms, etc. Moreover, note that some embodiments may be associated with a display of information to an operator.

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

1. A method comprising:
receiving a first image representative of a face of a subject from an image capture device;
determining one or more first facial geometry features from the first image;
comparing the one or more first facial geometry features to one or more corresponding target facial geometry features;
modulating the one or more first facial geometry features based upon the comparing of the one or more first facial geometry features to the one or more corresponding target facial geometry features to generate a first modulated image;
displaying the first modulated image in a display device;
receiving a second image of the face of the subject, the second image representative of a response of the subject to viewing the first modulated image; and
analyzing the second image to determine a mental process associated with the subject.

2. The method of claim 1, wherein analyzing the second image further comprises:
determining one or more second facial geometry features from the second image;
comparing the one or more second facial geometry features to the one or more corresponding target facial geometry features; and
determining the mental process of the subject based upon the comparing of the one or more second facial geometry features to the one or more corresponding target facial geometry features.

3. The method of claim 1, wherein modulating the one or more first facial geometry features includes morphing the first facial geometry features towards the one or more target facial geometry features by a predetermined amount.

4. The method of claim 1, wherein determining the one or more first facial geometry features further comprises:
extracting a shape mesh from the first image; and
identifying the one or more first facial geometry features from the shape mesh.

5. The method of claim 4, wherein the shape mesh includes a three-dimensional representation of an initial state of the subject.

6. The method of claim 4, wherein the modulating of the one or more first facial geometry features includes determining a target mesh for the subject associated with a target state of the face of the subject.

7. The method of claim 1, wherein the one or more facial geometry features includes at least one of an overall face geometry, a nose geometry, a mouth geometry, an eye geometry, and a brow geometry associated with the subject.

8. The method of claim 1, wherein the mental process is associated with a mental condition of the subject.

9. The method of claim 1, wherein the target facial geometry features are associated with a desired mental condition of the subject.

10. The method of claim 1, wherein the subject is a human subject.

11. A system to measure mental processes of a subject, comprising:
an image capture device;
a display device; and
a processing device, the processing device configured to:
receive a first image representative of a face of a subject from the image capture device;
determine one or more first facial geometry features from the first image;
compare the one or more first facial geometry features to one or more corresponding target facial geometry features;
modulate the one or more first facial geometry features based upon the comparing of the one or more first facial geometry features to the one or more corresponding target facial geometry features to generate a first modulated image;
display the first modulated image in the display device;
receive a second image of the face of the subject, the second image representative of a response of the subject to viewing the first modulated image; and
analyze the second image to determine a mental process associated with the subject.

12. The system of claim 11, wherein the processing device is further configured to:
determine one or more second facial geometry features from the second image;
compare the one or more second facial geometry features to the one or more corresponding target facial geometry features; and
determine the mental process of the subject based upon the comparing of the one or more second facial geometry features to the one or more corresponding target facial geometry features.

13. The system of claim 11, wherein modulating the one or more first facial geometry features includes morphing the first facial geometry features towards the one or more target facial geometry features by a predetermined amount.

14. The system of claim 11, wherein the processing device is further configured to:
extracting a shape mesh from the first image; and
identifying the one or more first facial geometry features from the shape mesh.

15. The system of claim 14, wherein the shape mesh includes a three-dimensional representation of an initial state of the subject.

16. A non-transitory, computer-readable medium storing instructions to be executed by a processor to perform a method comprising:
receiving a first image representative of a face of a subject from an image capture device;
determining one or more first facial geometry features from the first image;
comparing the one or more first facial geometry features to one or more corresponding target facial geometry features;
modulating the one or more first facial geometry features based upon the comparing of the one or more first facial geometry features to the one or more corresponding target facial geometry features to generate a first modulated image;
displaying the first modulated image in a display device;
receiving a second image of the face of the subject, the second image representative of a response of the subject to viewing the first modulated image; and
analyzing the second image to determine a mental process associated with the subject.

17. The medium of claim 16, wherein analyzing the second image further comprises:
determining one or more second facial geometry features from the second image;
comparing the one or more second facial geometry features to the one or more corresponding target facial geometry features; and
determining the mental process of the subject based upon the comparing of the one or more second facial geometry features to the one or more corresponding target facial geometry features.

18. The medium of claim 16, wherein modulating the one or more first facial geometry features includes morphing the first facial geometry features towards the one or more target facial geometry features by a predetermined amount.

19. The medium of claim 16, wherein determining the one or more first facial geometry features further comprises:
extracting a shape mesh from the first image; and
identifying the one or more first facial geometry features from the shape mesh.

20. The medium of claim 19, wherein the shape mesh includes a three-dimensional representation of an initial state of the subject.

21. A method comprising:
receiving a first image representative of a body portion of a subject from an image capture device;
determining one or more first geometry features from the first image;
comparing the one or more first geometry features to one or more corresponding target geometry features;
modulating the one or more first geometry features based upon the comparing of the one or more first geometry features to the one or more corresponding target geometry features to generate a first modulated image;
displaying the first modulated image in a display device;
receiving a second image of the body portion of the subject, the second image representative of a response of the subject to viewing the first modulated image; and
analyzing the second image to determine a mental process associated with the subject.

22. The method of claim 21, wherein analyzing the second image further comprises:
determining one or more second geometry features from the second image;
comparing the one or more second geometry features to the one or more corresponding target geometry features; and
determining the mental process of the subject based upon the comparing of the one or more second geometry features to the one or more corresponding target geometry features.

23. The method of claim 21, wherein modulating the one or more first geometry features includes morphing the first geometry features towards the one or more target geometry features by a predetermined amount.

24. The method of claim 21, wherein the body portion includes at least one of a face of the subject or a portion of an arm of the subject.
